(19) United States
(12) Patent Application Publication
(10) Pub. No.: US 2024/0278029 A1
(43) Pub. Date: Aug. 22, 2024

(54) LASER RIBBON BONDING FOR BATTERY AND CAPACITOR

(71) Applicant: Cardiac Pacemakers, Inc., St. Paul, MN (US)

(72) Inventors: James Michael English, Cahir (IE); Moira B. Sweeney, St. Paul, MN (US); Robert O' Donoghue, Clonmel (IE); Benjamin J. Haasl, Forest Lake, MN (US)

(21) Appl. No.: 18/441,973

(22) Filed: Feb. 14, 2024

Related U.S. Application Data
(60) Provisional application No. 63/446,938, filed on Feb. 20, 2023.

(51) Int. Cl. A61N 1/39; H01M 50/213; H01M 50/224; H01M 50/247; H01M 50/545; H01M 50/562; H01R 4/02; H01R 43/02

(52) U.S. Cl. CPC A61N 1/3956; H01M 50/213; H01M 50/224; H01M 50/247; H01M 50/545; H01M 50/562; H01R 4/023; H01R 43/0221; H01M 2220/30; H01R 2201/12

(57) ABSTRACT

An ambulatory medical device includes an energy storage component having a metal case and one output pin; a circuit board including a supply connection and a ground connection; a first conductive wire connected to the supply connection of the circuit board and the output pin of the energy storage component; and a second conductive wire connected to the ground connection of the circuit board and the metal case of the energy storage component.

### TECHNICAL FIELD

This patent application relates to implantable medical devices and, in particular, to techniques to include a battery or a capacitor in implantable devices.

### BACKGROUND

Ambulatory medical devices can be implantable and can include a hermetically sealed metal case or metal housing. The electronics of an implantable device are contained within the metal housing. Typically, an implantable medical device is battery powered and the battery is within the housing. Some ambulatory medical devices can provide electrical defibrillation therapy and include one or more capacitors capable of delivering high voltage defibrillation energy. Packaging of the electronic components of ambulatory medical devices within the metal housing can be challenging.

### SUMMARY

Systems and methods are disclosed relating to ambulatory medical devices and to their assembly. Example 1 includes subject matter (such as an implantable medical device) including an energy storage component having a metal case and one output pin; a circuit board including a supply connection and a ground connection; a first conductive wire connected to the supply connection of the circuit board and the output pin of the energy storage component; and a second conductive wire connected to the ground connection of the circuit board and the metal case of the energy storage component.

In Example 2, the subject matter of Example 1 optionally includes an energy storage component that is a battery, and the circuit board includes one or more circuit components that receive electrical energy from the battery.

In Example 3, the subject matter of Example 1 optionally includes the implantable medical device is configured to provide electrical defibrillation therapy, and the energy storage component is a capacitor to store electrical energy used for the defibrillation therapy; and the circuit board includes one or more circuit components to charge the capacitor.

In Example 4, the subject matter of one or any combination of Examples 103 optionally includes the metal case of the energy storage component including a flat surface, and the second conductive wire is a metal ribbon connected to the ground connection of the circuit board and laser-bonded to the flat surface of the metal case.

In Example 5, the subject matter of one or any combination of Examples 1˜4 optionally includes the metal case of the energy storage component including a curved surface, and the second conductive wire is a metal ribbon connected to the ground connection of the circuit board and laser-bonded to the curved surface of the metal case.

In Example 6, the subject matter of one or any combination of Examples 1-5 optionally includes the metal case of the energy storage component including a first side, a second side, and an indent on the first side, and the indent includes a flat indented surface at a height between the first side and the second side of the energy storage component. The second conductive wire is a metal ribbon connected to the ground connection of the circuit board and laser-bonded to the flat indented surface of the metal case.

In Example 7, the subject matter of one or any combination of Examples 1-6 optionally includes the one output pin of the energy storage device including a feedthrough including a feedthrough pin and a sleeve around the feedthrough pin; and the first conductive wire including a metal ribbon connected to the supply connection of the circuit board and laser-bonded to the sleeve.

In Example 8, the subject matter of one or any combination of Examples 1-7 optionally includes the feedthrough pin including lithium and the sleeve around the feedthrough pin includes nickel

Example 9 includes subject matter (such as a method of forming an implantable medical device) or can optionally be combined with one or any combination of Examples 1-8 to include such subject matter, including connecting a first end of a first conductive wire to a supply connection of a circuit board of the implantable medical device and laser bonding a second end of the first conductive wire to an only pin of the energy storage component; and connecting a first end of a second conductive wire to a ground connection of the circuit board and laser bonding a second end of the second conductive wire to a metal case of the energy storage component.

In Example 10, the subject matter of Example 9 optionally includes mounting one or more circuit components on the circuit board; connecting a pin of a battery having only one pin to the supply connection to supply energy to the one or more circuit components of the circuit board; and laser bonding the second end of the second conductive wire to a metal case of the battery.

In Example 11, the subject matter of Example 9 optionally includes mounting one or more circuit components on the circuit board; connecting a pin of a capacitor having only one pin to the circuit board; and laser bonding the second end of the second conductive wire to the metal case of the capacitor.

In Example 12, the subject matter of one or any combination of Examples 9-11 optionally includes laser bonding the second end of the second conductive wire to a flat surface of the metal case of the energy storage component.

In Example 13, the subject matter of one or any combination of Examples 9-12 optionally includes laser bonding the second end of the second conductive wire to a curved surface of the metal case of the energy storage component.

In Example 14, the subject matter of one or any combination of Examples 9-13 optionally includes forming the metal case of the energy storage component to have a flat indented surface between a first side of the energy storage component and a second side of the energy storage component such that the flat indented surface is parallel to the first side of the energy storage component; and laser bonding the second end of the second conductive wire to the flat indented surface of the metal case of the energy storage component.

In Example 15, the subject matter of one or any combination of Examples 9-14 optionally includes forming a feedthrough for the energy storage device; forming the one output pin of the energy storage device as a feedthrough pin; forming a sleeve around the feedthrough pin; and wherein the first conductive wire includes a metal ribbon connected to the supply connection of the circuit board and laser-bonded to the sleeve of the feedthrough pin.

In Example 16, the subject matter of one or any combination of Examples 9-15 optionally includes forming a feedthrough pin that includes lithium; and forming the sleeve to include nickel and exclude gold.

Example 17 includes subject matter (such as an implantable medical device) or can optionally be combined with one or any combination of Examples 1-16 to include such subject matter, including a battery having a metal battery case and including one battery pin; a capacitor having a metal capacitor case and including one capacitor pin, the capacitor to store charge used for defibrillation provided by the implantable medical device; a circuit board including a supply connection and at least one ground connection; a first metal ribbon connected to the at least one ground connection of the circuit board and laser-bonded to the metal battery case; a second metal ribbon a third conductive wire connected to the supply connection of the circuit board and laser-bonded to the battery pin; and a third metal ribbon connected to the at least one ground connection of the circuit board and laser-bonded to the metal capacitor case.

In Example 18, the subject matter of Example 17 optionally includes one or more circuit components on the circuit board that receive electrical energy from the battery and charge the battery capacitor; and a fourth metal ribbon laser-bonded to the capacitor pin and connected to the circuit board.

In Example 19, the subject matter of one or both of Examples 17 and 18 optionally includes the metal battery case and the capacitor metal case each including a curved surface, and the first metal ribbon and the third metal ribbon are respectively laser-bonded to the curved surface of the metal battery case and the curved surface of the metal capacitor case.

In Example 20, the subject matter of one or any combination of Examples 17-19 optionally includes the metal battery case and the capacitor metal case each including a flat surface facing away from the circuit board, and the first metal ribbon and the third metal ribbon are respectively laser bonded to the flat surface of the metal battery case and the flat surface of the metal capacitor case.

The non-limiting Examples can be combined in any permutation or combination. This summary is intended to provide an overview of the subject matter of the present application. It is not intended to provide an exclusive or exhaustive explanation of the invention. The detailed description is included to provide further information about the subject matter of the present patent application.

### BRIEF DESCRIPTION OF THE DRAWINGS

FIG. 1 is an illustration of portions of a system that uses an ambulatory medical device.

FIG. 2 is a view of another example of an ambulatory medical device.

FIG. 3 is a view of a portion of an example of an ambulatory medical device.

FIG. 4 shows an example of an approach to incorporating a battery into an assembly of an ambulatory medical device.

FIG. 5 shows an example of another approach to incorporating a battery into an assembly of an ambulatory medical device.

FIG. 6 is a view of a battery pin.

FIG. 7 is a view of a portion of a capacitor of an ambulatory medical device.

FIG. 8 is a flow diagram of an example of a method of manufacture for a portion of an ambulatory medical device.

FIG. 9 shows an example of another approach to incorporating a battery into an assembly of an ambulatory medical device.

### DETAILED DESCRIPTION

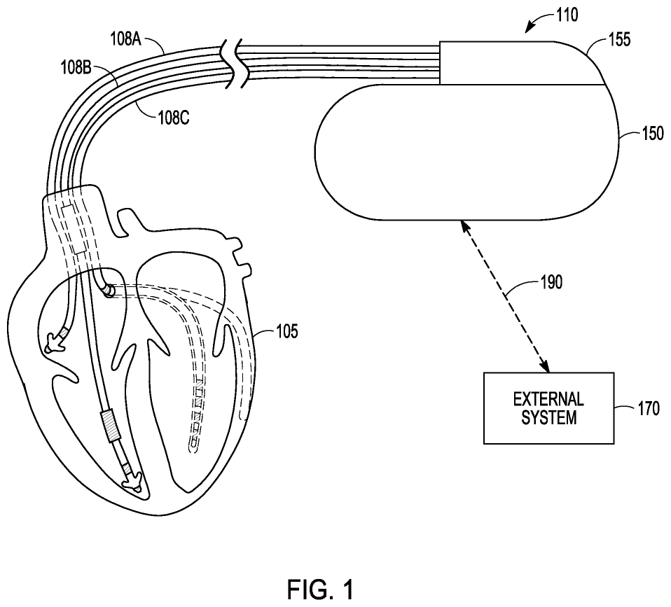

This document relates to techniques to assemble an energy storage component (e.g., a battery or a capacitor) in an ambulatory medical device. FIG. 1 is an illustration of portions of a system that uses an implantable medical device (IMD). Some examples of the IMD 110 include a pacemaker, a defibrillator, a cardiac resynchronization therapy (CRT) device, a combination of such devices, or a diagnostic-only device. The system also typically includes an IMD programmer or other external device 170 that communicates wireless signals 190 with the IMD 110, such as by using radio frequency (RF) or other telemetry signals.

The IMD 110 can be coupled by one or more conductive leads 108A-C to heart 105. The cardiac leads 108A-C in the example of FIG. 1 include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals. Sensed electrical cardiac signals can be sampled to create an electrogram. An electrogram can be analyzed by the IMD and/or can be stored in the IMD and later communicated to an external device where the sampled signals can be displayed for analysis.

The cardiac leads 108A-C include right atrial (RA) lead 108A, right ventricle (RV) lead 108B, and a third cardiac lead 108C for placement in a coronary vein lying epicardially on the left ventricle (LV) via the coronary vein.

The IMD 110 includes a hermetically-sealed metal housing or metal case 150 that houses electronic circuits and a header connector 155. Conductive leads (e.g., cardiac leads 108A-C) are connected to the IMD through the header connector 155. The header connector 155 includes lead receptacles for the cardiac leads 108A-C. The lead receptacles can be encapsulated in a mold material (e.g., an epoxy).

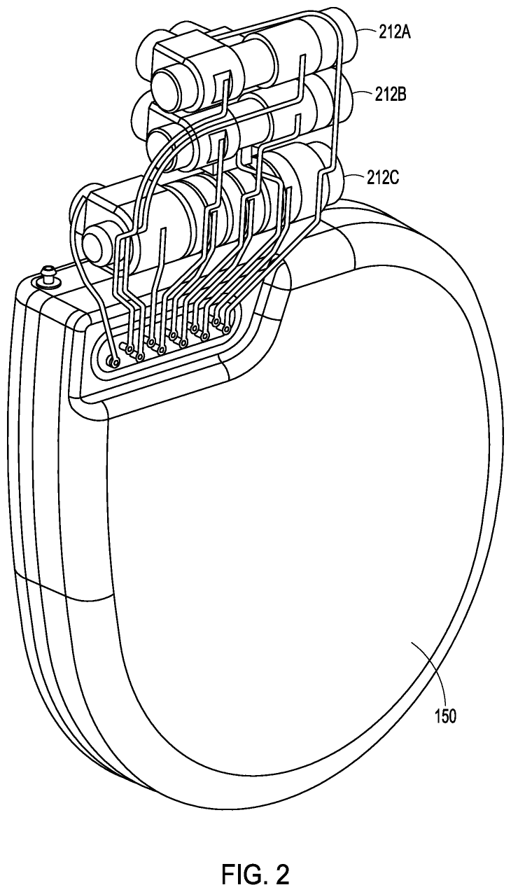

FIG. 2 is a view of another example of a medical device 110. The view shows the metal housing 150 of the medical device and shows the header connector without the mold material to expose the lead receptacles 212A-C.

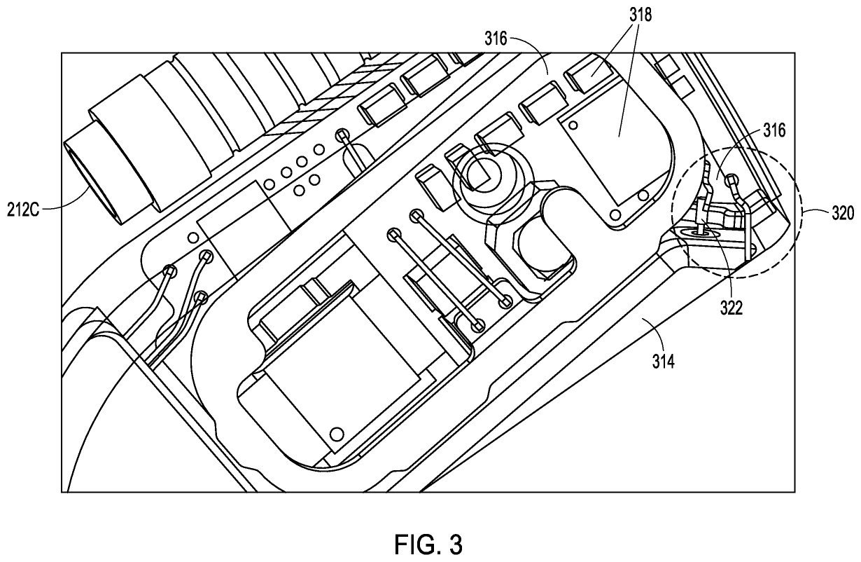

FIG. 3 is a view of a portion of the medical device 110. A side plate of the metal housing 150 is removed to expose some of the electronics of the medical device 110 including a portion of a battery 314 and a circuit board 316. The circuit board 316 may be a printed circuit board (PCB) or a ceramic circuit board and includes circuit components 318 mounted on the circuit board 316. The battery 314 provides a circuit supply for circuit components 318.

The dashed circle 320 in FIG. 3 shows the connection of the battery 314 to the circuit board 316. The battery 314 includes one pin 322 and a metal battery case. The battery pin 322 may be the positive output terminal of the battery 314 and the battery case may be ground or a return connection for the battery 314. The assembly in FIG. 3 shows two conductive wires that are metal ribbon (e.g., copper ribbon). One metal ribbon connects the supply connection of the circuit board 316 to the battery pin 322. The metal ribbon is laser-bonded to battery pin. In certain examples, the metal ribbon is laser-bonded to the supply connection of the circuit board 316. The other metal ribbon is connects the ground connection of the circuit board 316 to the battery case. This metal ribbon is laser-bonded to the battery case and in certain examples, the metal ribbon is laser-bonded to the ground connection of the circuit board 316. In certain examples, the connections to the supply and ground of the circuit board 316 are soldered connections.

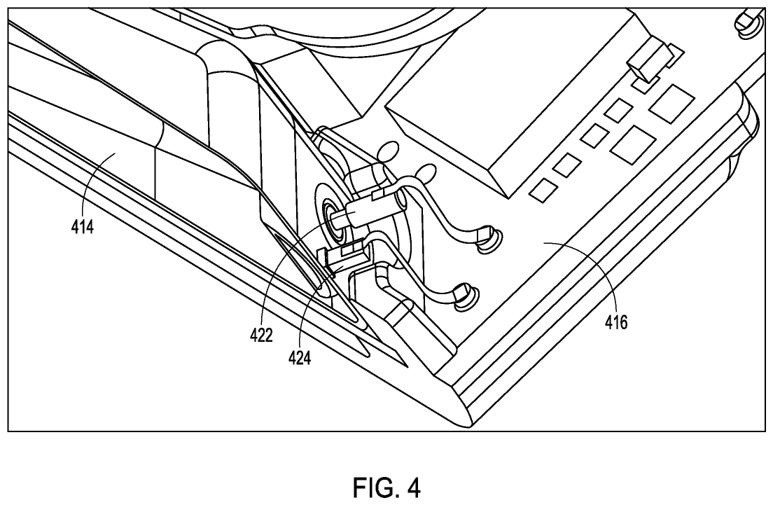

FIG. 4 shows another approach to incorporating a battery into an assembly of a medical device. The battery 414 includes two terminals. One terminal is a positive terminal 422 and the other terminal a negative terminal 424 of the battery 314. The wires are connected to the terminals using solder. Typically, the soldering requires a gold coating to be applied to the two terminals or studs of the battery so they can be soldered.

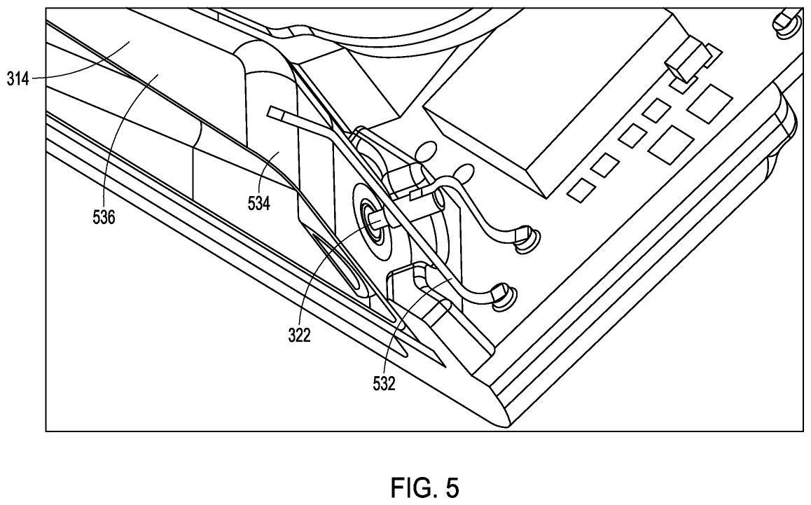

FIG. 5 shows another view of the battery connection approach of FIG. 3. The gold coated negative terminal is removed. The one battery pin 322 does not have to be coated with gold because laser bonding is used to connect to the battery pin 322.

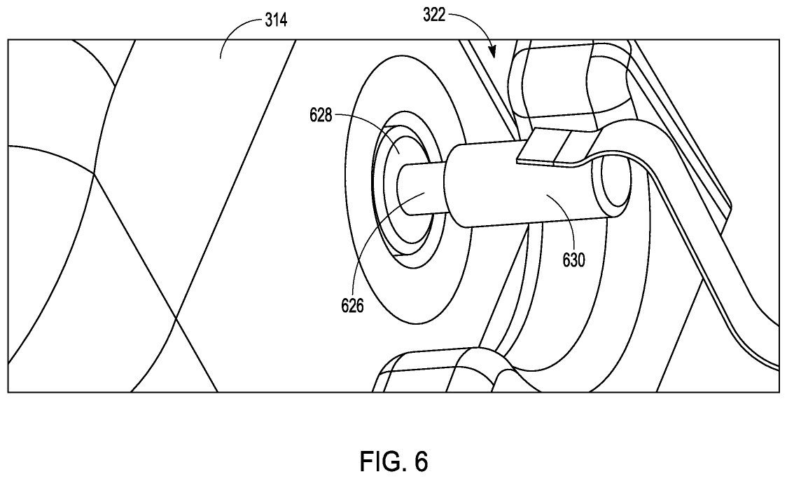

FIG. 6 is a view of the battery pin 322. The battery pin 322 is a feedthrough assembly that includes a pin 626, an insulator 628 to insulate the pin from the case of the battery 314, and a sleeve 630 over the pin 626. In some examples, the pin 626 of the feedthrough includes lithium, the insulator 628 includes glass, and the sleeve 630 includes nickel. A metal ribbon 632 is laser bonded to the sleeve 630. Because the metal ribbon 632 is laser-bonded instead of soldered, the sleeve 630 does need to be coated with gold. Because the battery pin 322 does not need a gold coating and because the negative terminal is not needed, the battery 314 of FIG. 6 can be less expensive than the battery 414 of FIG. 4.

Returning to FIG. 5, the metal case of the battery 314 includes a curved surface 534 having a radius, and a flat top surface 536 that faces away from the circuit board. The metal ribbon 532 is shown laser-bonded to the curved surface 534 of the battery case. In some examples, the metal ribbon 532 is laser-bonded to the top surface 536 of the metal case of the battery 314.

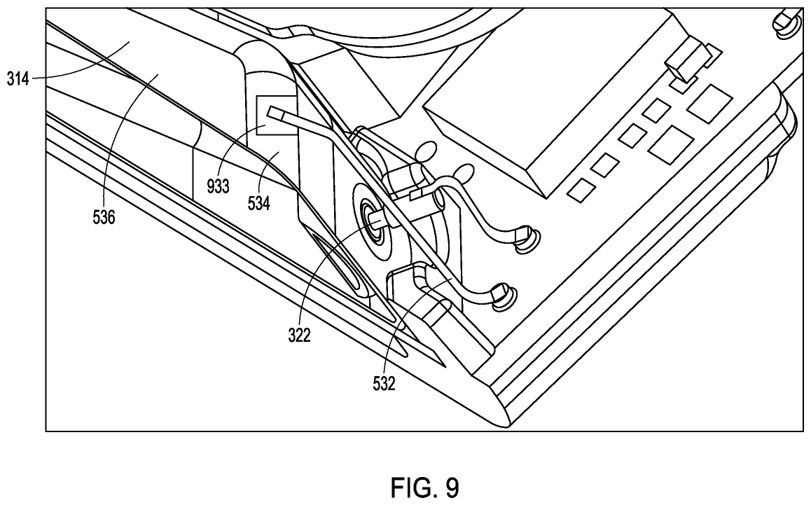

FIG. 9 shows another view of the battery connection approach of FIG. 3. The battery case includes an indent 933 on the top surface 536. The indent has a flat indented surface at a height below the top of the top flat surface 536. The metal ribbon 532 can be bonded to the flat indented surface. The depth of the indent may be sized to fit the height of the metal ribbon so that the laser-bonded metal ribbon does not add to the height of the battery case.

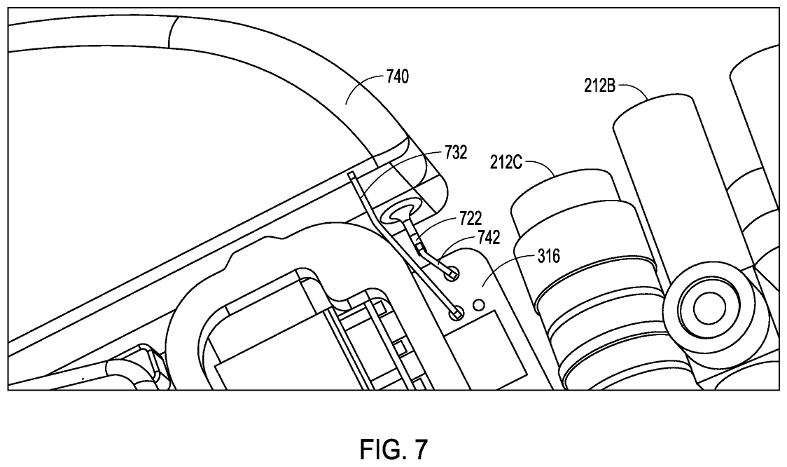

FIG. 7 is a view of a portion of a capacitor 740 of the implantable medical device 110 and the circuit board 316. The capacitor 740 can be used to store charge used for defibrillation provided by the implantable medical device. One or more of the circuit components 318 of the circuit board can be used to charge the capacitor 740. The capacitor includes one capacitor pin 722. The capacitor pin 722 is one terminal (e.g., the positive terminal) of the capacitor 740 and the capacitor case is the other terminal (e.g., the ground terminal) of the capacitor 740.

A conductive wire 742 (e.g., a metal ribbon) connects the capacitor pin 722 to the circuit board 316 and another conductive wire 732 connects the case of the capacitor to a ground connection of the circuit board 316 that can connect the capacitor 740 to a ground plane of the circuit board 316. Conductive wire 742 can be laser-bonded to the capacitor pin 722 and conductive wire 732 can be laser-bonded to the capacitor case. Conductive wire 732 can be laser-bonded to a curved surface of the capacitor case, a flat surface of the capacitor case, or a surface of an indent (not shown) formed on the surface of the metal capacitor case.

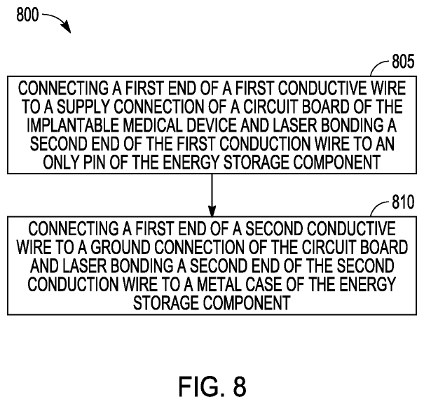

FIG. 8 is a flow diagram of an example of a method 800 of manufacture of a portion of an implantable medical device. The method 800 is a technique to incorporate an energy storage component (such as a battery or a capacitor) into an ambulatory medical device, such as the implantable medical device 110 of FIG. 1.

The energy storage component has only one pin. At block 805, one end of a first conductive wire is connected to a supply connection of a circuit board of the implantable medical device and the other end of the first conductive wire is laser-bonded to the one pin of the energy storage component. In some examples, both ends are laser-bonded, and the first conductive wire is laser-bonded to the supply connection of the circuit board.

In some examples, the energy storage component is a battery and the one pin of the energy storage component is a battery pin. The method includes forming a feedthrough of the battery that includes a battery pin insulated (e.g., with glass) from the metal case of the battery. A metal sleeve is formed around the battery pin, and the conductive wire is laser-bonded to the metal sleeve. In some examples, the sleeve includes nickel. A metal that improves soldering (e.g., gold) can be excluded from the metal sleeve because soldering is not used to connect to the battery. The method 800 can include mounting circuit components on the circuit board and connecting the battery pin to the supply connection of the circuit board supplies electrical energy to the circuit components.

In some examples, the energy storage component is a capacitor the one pin of the energy storage component is a capacitor pin. The capacitor is sized to store charge for defibrillation therapy provided by the medical device. Connecting the capacitor pin to the circuit board can connect the capacitor to circuit components of the circuit board that charge the capacitor.

At block 810, one end of a second conductive wire is connected to a ground connection of the circuit board and the other end of the second conductive wire is laser-bonded to a metal case of the energy storage component. In some examples, the conductive wires are metal ribbon wires. The conductive wire can be laser-bonded to a top surface of the energy storage component or a curved surface just below the top surface. Laser-bonding to a point below the top surface does not add to the height of the energy storage component. In some examples, the metal case is formed to have a flat surface at a height lower than the top surface of the energy storage component. In some examples, both ends of the conductive wires are laser-bonded and the connections to the circuit board are laser bonds.

The techniques described herein reduce complexity and cost of incorporating a battery or a defibrillation capacitor into an ambulatory medical device.

### Additional Description

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An implantable medical device, the device comprising:
an energy storage component having a metal case and one output pin;
a circuit board including a supply connection and a ground connection;
a first conductive wire connected to the supply connection of the circuit board and the output pin of the energy storage component; and
a second conductive wire connected to the ground connection of the circuit board and the metal case of the energy storage component.

2. The device of claim 1, wherein the energy storage component is a battery, and the circuit board includes one or more circuit components that receive electrical energy from the battery.

3. The device of claim 1,
wherein the device is configured to provide electrical defibrillation therapy, and the energy storage component is a capacitor to store electrical energy used for the defibrillation therapy; and
wherein the circuit board includes one or more circuit components to charge the capacitor.

4. The device of claim 1, wherein the metal case of the energy storage component includes a flat surface, and the second conductive wire is a metal ribbon connected to the ground connection of the circuit board and laser-bonded to the flat surface of the metal case.

5. The device of claim 1, wherein the metal case of the energy storage component includes a curved surface, and the second conductive wire is a metal ribbon connected to the ground connection of the circuit board and laser-bonded to the curved surface of the metal case.

6. The device of claim 1,
wherein the metal case of the energy storage component includes a first side, a second side, and an indent on the first side, the indent includes a flat indented surface at a height between the first side and the second side of the energy storage component; and
wherein the second conductive wire is a metal ribbon connected to the ground connection of the circuit board and laser-bonded to the flat indented surface of the metal case.

7. The device of claim 1,
wherein the one output pin of the energy storage device includes a feedthrough including a feedthrough pin and a sleeve around the feedthrough pin; and
wherein the first conductive wire includes a metal ribbon connected to the supply connection of the circuit board and laser-bonded to the sleeve.

8. The device of claim 1, wherein the feedthrough pin includes lithium and the sleeve around the feedthrough pin includes nickel.

9. A method of making a portion of an implantable medical device, the method comprising:
connecting a first end of a first conductive wire to a supply connection of a circuit board of the implantable medical device and laser bonding a second end of the first conductive wire to an only pin of the energy storage component; and
connecting a first end of a second conductive wire to a ground connection of the circuit board and laser bonding a second end of the second conductive wire to a metal case of the energy storage component.

10. The method of claim 9, including:
mounting one or more circuit components on the circuit board;
wherein the connecting the first end of a first conductive wire to the supply connection of the circuit board includes connecting a pin of a battery having only one pin to the supply connection to supply energy to the one or more circuit components of the circuit board; and
wherein the laser bonding the second end of the second conductive wire includes laser bonding the second end of the second conductive wire to a metal case of the battery.

11. The method of claim 9, including:
mounting one or more circuit components on the circuit board;
wherein the connecting the first end of a first conductive wire to the supply connection of the circuit board includes connecting a pin of a capacitor having only one pin to the circuit board; and
wherein the laser bonding the second end of the second wire includes laser bonding the second end of the second conductive wire to the metal case of the capacitor.

12. The method of claim 9, wherein the laser bonding the second end of the second conductive wire includes laser bonding the second end of the second conductive wire to a flat surface of the metal case of the energy storage component.

13. The method of claim 9, wherein the laser bonding the second end of the second conductive wire includes laser bonding the second end of the second conductive wire to a curved surface of the metal case of the energy storage component.

14. The method of claim 9, including:
forming the metal case of the energy storage component to have a flat indented surface between a first side of the energy storage component and a second side of the energy storage component, wherein the flat indented surface is parallel to the first side of the energy storage component; and
wherein the laser bonding the second end of the second conductive wire includes laser bonding the second end of the second conductive wire to the flat indented surface of the metal case of the energy storage component.

15. The method of claim 9, including:
forming a feedthrough for the energy storage device;
forming the one output pin of the energy storage device as a feedthrough pin;
forming a sleeve around the feedthrough pin; and
wherein the first conductive wire includes a metal ribbon connected to the supply connection of the circuit board and laser-bonded to the sleeve of the feedthrough pin.

16. The device of claim 9, wherein the forming the one output pin includes forming a feedthrough pin that includes lithium; and
wherein forming the sleeve includes forming the sleeve to include nickel and exclude gold.

17. An implantable medical device comprising:
a battery having a metal battery case and including one battery pin;
a capacitor having a metal capacitor case and including one capacitor pin, the capacitor to store charge used for defibrillation provided by the implantable medical device;
a circuit board including a supply connection and at least one ground connection;
a first metal ribbon connected to the at least one ground connection of the circuit board and laser-bonded to the metal battery case;
a second metal ribbon a third conductive wire connected to the supply connection of the circuit board and laser-bonded to the battery pin; and
a third metal ribbon connected to the at least one ground connection of the circuit board and laser-bonded to the metal capacitor case.

18. The implantable medical device of claim 17, including:
one or more circuit components on the circuit board that receive electrical energy from the battery and charge the battery capacitor; and
a fourth metal ribbon laser-bonded to the capacitor pin and connected to the circuit board.

19. The implantable medical device of claim 17, wherein the metal battery case and the capacitor metal case each include a curved surface, and the first metal ribbon and the third metal ribbon are respectively laser-bonded to the curved surface of the metal battery case and the curved surface of the metal capacitor case.

20. The implantable medical device of claim 17, wherein the metal battery case and the capacitor metal case each include a flat surface facing away from the circuit board, and the first metal ribbon and the third metal ribbon are respectively laser bonded to the flat surface of the metal battery case and the flat surface of the metal capacitor case.
